(19) United States
(12) Patent Application Publication
(10) Pub. No.: US 2021/0001003 A1
(43) Pub. Date: Jan. 7, 2021

(54) WOUND-TREATING ABSORBENT

(71) Applicants: BAXTER INTERNATIONAL INC., Deerfield, IL (US); BAXTER HEALTHCARE SA, Glattpark (Opfikon) (CH)

(72) Inventors: Vadim Valerievich KRONGAUZ, Bartlett, IL (US); Wei XIE, Buffalo Grove, IL (US); Michael Tung Kiung LING, I, Vernon Hills, IL (US)

(21) Appl. No.: 16/767,005

(22) PCT Filed: Nov. 27, 2018
(86) PCT No.: PCT/US2018/062513

§ 371 Date: May 26, 2020

Related U.S. Application Data
(60) Provisional application No. 62/591,481, filed on Nov. 28, 2017.

(51) Int. Cl. A61L 26/00

(52) U.S. Cl. CPC A61L 26/0066; A61L 26/0023; A61L 26/0042; A61L 26/008; A61L 2300/802; A61L 2300/254; A61L 2300/414; A61L 2300/406; A61L 2300/428; A61L 2400/04

(57) ABSTRACT

The present disclosure provides wound-treating absorbent kits that comprise a set of hemostatic compositions including at least (1) a first hemostatic composition including a first crosslinked polysaccharide selected from the group consisting of cyclodextrin and dextran, and (2) a second hemostatic composition including a second crosslinked polysaccharide selected from the group consisting of cyclodextrin and dextran. In some embodiments, the first hemostatic composition has a first degree of crosslinking, and the second hemostatic composition has a second degree of crosslinking higher than the first degree of crosslinking. Also provided are methods of treating a wound by selecting a hemostatic composition from the disclosed set of hemostatic compositions, and administering the selected hemostatic composition to a site of the wound.

### PRIORITY CLAIM

This application claims priority to U.S. Provisional Application No. 62/591,481, filed on Nov. 28, 2017, the entire contents of which are incorporated herein.

### BACKGROUND

Hemostatic agents and sealants are currently used as an aid to stop bleeding, including hemorrhaging, during surgery. The FDA has approved hemostatic matrices, such as FLOSEAL® (Baxter International), for use in patients to augment the natural clotting cascade or to mechanically stop bleeding at a surgical or wound site. FLOSEAL® is a flowable product comprising gelatin and thrombin. The thrombin is first reconstituted with sodium chloride, and then mixed with the gelatin matrix component for use in a syringe.

Gelatin is derived from animal products such as tendon collagen and skin. Due to concerns about allergies to materials of bovine origin, certain hemostatic compositions that are not of animal origin, such as polyanhydroglucuronic acid, are of interest.

### SUMMARY

The present disclosure provides a wound-treating absorbent kit comprising a set of hemostatic compositions including at least (1) a first hemostatic composition including a first crosslinked polysaccharide selected from the group consisting of cyclodextrin and dextran, and (2) a second hemostatic composition including a second crosslinked polysaccharide selected from the group consisting of cyclodextrin and dextran. In some embodiments, the first hemostatic composition has a first degree of crosslinking, and the second hemostatic composition has a second degree of crosslinking higher than the first degree of crosslinking.

Currently, many hemostatic compositions used in patients comprise an animal derived starting material (e.g., gelatin) to manufacture a hemostatic matrix. The present compositions use non-animal polysaccharide/starch polymers to make the hemostatic matrix.

In each or any of the above- or below-mentioned embodiments, each of the first and second hemostatic compositions may include crosslinked β-cyclodextrin.

In each or any of the above- or below-mentioned embodiments, each of the first and second hemostatic compositions may be in powdered form.

In each or any of the above- or below-mentioned embodiments, the wound-treating absorbent kit may include a pharmaceutically acceptable diluent for reconstitution of any of the first and second hemostatic compositions.

In each or any of the above- or below-mentioned embodiments, each of the first and second hemostatic compositions may include a respective crosslinking agent selected from the group consisting of diglycidyl ether, epichlorohydrin, diisocyanate, dicarboxylic acid chlorides, dicarboxylic acid, acid anhydrides, poly(d,l-lactic acid), citric acid, glycerol, dialdehydes, diacyl chlorides, and epoxides.

In each or any of the above- or below-mentioned embodiments, the first and second hemostatic compositions may differ from each other in at least one of an amount and a type of the respective crosslinking agents.

In each or any of the above- or below-mentioned embodiments, each of the first and second hemostatic compositions may have a swelling capability from about 38% to about 1600%.

In each or any of the above- or below-mentioned embodiments, the second hemostatic composition may be configured to absorb less fluid than the first hemostatic composition.

In each or any of the above- or below-mentioned embodiments, the wound-treating absorbent kit may include at least one additive selected from the group consisting of water-soluble antimicrobial medicines, enzymes, and growth factor agents.

In each or any of the above- or below-mentioned embodiments, any of the first and second hemostatic compositions may be mixed with at least one agent selected from the group consisting of a blood clotting factor, fibrin, an antiseptic agent, an anti-microbial agent, a vitamin, a micronutrient, an antibiotic agent, and an antifungal agent.

The present disclosure also provides a method of treating a wound. The method includes selecting a hemostatic composition from a set of hemostatic compositions comprising at least (1) a first hemostatic composition including a first crosslinked polysaccharide selected from the group consisting of cyclodextrin and dextran, wherein the first hemostatic composition has a first degree of crosslinking, and (2) a second hemostatic composition including a second crosslinked polysaccharide selected from the group consisting of cyclodextrin and dextran, wherein the second hemostatic composition has a second degree of crosslinking higher than the first degree of crosslinking. The selected hemostatic composition is administered to a site of the wound.

In each or any of the above- or below-mentioned embodiments, the hemostatic composition may be selected according to a desired swelling capability.

In each or any of the above- or below-mentioned embodiments, the hemostatic composition may be selected according to a reaction parameter selected from the group consisting of a reaction time, a reaction temperature, and a combination thereof

In each or any of the above- or below-mentioned embodiments, the selected hemostatic composition may be applied in powder form.

In each or any of the above- or below-mentioned embodiments, any of the first and second hemostatic compositions may be mixed with at least one agent selected from the group consisting of a blood clotting factor, fibrin, an antiseptic agent, an anti-microbial agent, a vitamin, a micronutrient, an antibiotic agent, an antifungal agent, prior to being administered to the site of the wound.

In each or any of the above- or below-mentioned embodiments, at least one additive selected from the group consisting of water-soluble antimicrobial medicines, enzymes, and growth factor agents may be administered with the selected hemostatic composition.

It is accordingly an advantage of the present disclosure to provide a wound-treating absorbent kit with a set of hemostatic compositions having a high degree of water absorption and capable of rapid expansion for filling the bleeding wounds.

It is a further advantage of the present disclosure to provide a method in which the extent of water absorption and expansion can be controlled by a change in the degree of crosslinking.

Additional features and advantages of the disclosed kits and methods are described in, and will be apparent from, the following Detailed Description and the figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1 is a graph showing kinetics of water absorption by crosslinked β-cyclodextrin polymers according to an embodiment of the present disclosure.

FIG. 2 is a graph showing kinetics of water absorption by FLOSEAL and Sephadex® G-10, G-25, G-50, G-75 crosslinked dextran based polymers according to embodiments of the present disclosure.

FIG. 3 is a graph showing the extent of swelling caused by water absorption versus the degree of crosslinking of hydrophilic polymers according to embodiments of the present disclosure.

### DETAILED DESCRIPTION

The present disclosure provides a wound-treating absorbent kit comprising a set of hemostatic compositions including at least (1) a first hemostatic composition including a first crosslinked polysaccharide selected from the group consisting of cyclodextrin and dextran, and (2) a second hemostatic composition including a second crosslinked polysaccharide selected from the group consisting of cyclodextrin and dextran. The hemostatic composition may be selected according to a desired swelling capability. For example, the extent of water absorption and expansion can be controlled by a change in the degree of crosslinking.

Unless otherwise defined, all technical and scientific terms used herein have the same meaning as commonly understood by one of ordinary skill in the art to which the present disclosure belongs. The following references provide one of skill with a general definition of many of the terms used in this disclosure: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

As used in the present disclosure and the appended claims, the terms “a”, “an” and “the” include plural reference as well as singular reference unless the context clearly dictates otherwise.

As used herein, the following terms have the meanings ascribed to them unless specified otherwise.

The term “about” or “approximately” means an acceptable error for a particular value as determined by one of ordinary skill in the art, which depends in part on how the value is measured or determined. In certain embodiments, the term “about” or “approximately” means within 1, 2, 3 or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5% or 0.1% of a given value or range. Whenever the term “about” or “approximately” precedes the first numerical value in a series of two or more numerical values, it is understood that the term “about” or “approximately” applies to each one of the numerical values in that series.

A “hemostatic composition” refers to a composition useful to stop or reduce bleeding that results from injury or surgery, and/or to promote the coagulation cascade. A “flowable” composition or “hydrogel” refers to a substantially liquid, slightly viscous solution, solid, semi-solid solid, pseudoplastic, or plastic structure containing an aqueous component to produce a gelatinous or jelly-like mass, or paste-like solution that has the properties of being able to flow through a syringe or other device and be administering to a subject. The flowable hydrogel is a liquid-like, slightly viscous solution, or paste-like solution at room temperature and body temperature. In some embodiments, a flowable composition is one that holds shape when extruded through a syringe or other device for administering to a subject.

The wound-treating absorbent kit of the present disclosure includes a set of hemostatic compositions including at least (1) a first hemostatic composition including a first crosslinked polysaccharide selected from the group consisting of cyclodextrin and dextran, and (2) a second hemostatic composition including a second crosslinked polysaccharide selected from the group consisting of cyclodextrin and dextran. Cyclodextrins are of three types: α-cyclodextrin, β-cyclodextrin, and γ-cyclodextrin. α-, β-, and γ-cyclodextrins are composed of six, seven, and eight α-(1,4)-linked glucose units, respectively. In terms of properties, cyclodextrin has a hydrophilic outer surface and a lipophilic central cavity. In some embodiments, each of the first and second hemostatic compositions may include crosslinked β-cyclodextrin. In some embodiments, at least one of the first and second hemostatic compositions includes cross-linked dextran. Dextran has a chain length of 3-2000 kilodaltons.

In some embodiments, each of the first and second hemostatic compositions can be crosslinked through carboxylic groups, forming a gel molecule, which is readily capable of polar fluids sorption accompanied by swelling. In some embodiments, the crosslinking agent is selected from the group consisting of diglycidyl ether, epichlorohydrin, diisocyanate, dicarboxylic acid chlorides, dicarboxylic acid, acid anhydrides, poly(d,l-lactic acid), citric acid, glycerol, dialdehydes, diacyl chlorides, and epoxides.

In some embodiments, the wound-treating absorbent kit may include at least one additive selected from the group consisting of water-soluble antimicrobial medicines, enzymes, and growth factor agents. In some embodiments, any of the first and second hemostatic compositions may be mixed with at least one agent selected from the group consisting of a blood clotting factor, fibrin, an antiseptic agent, an anti-microbial agent, a vitamin, a micronutrient, an antibiotic agent, and an antifungal agent.

In some embodiments, each of the first and second hemostatic compositions may be in powdered form. In some embodiments, most of the particles contained in the powdered hemostatic compositions (e.g., more than 50% w/w, more than 80%, or more than 90% w/w) may have particle sizes of from 10 to 1000 μm, from 50 to 800 μm, from 50 to 700 μm, from 150 to 700 μm, from 200 to 700 μm, from 300 to 550 μm, or from 350 to 550 μm. In the powdered form, the hemostatic composition may be storage-stable for a long time even at elevated temperatures (e.g., more than 20° C., more than 30° C., or even more than 40° C.). In some embodiments, the hemostatic compositions have a moisture content of below 15% (w/w), below 10%, below 5%, or below 1%.

In some embodiments, the wound-treating absorbent kit may include a pharmaceutically acceptable diluent for reconstitution of any of the first and second hemostatic compositions. The powdered hemostatic composition according to the present disclosure can rapidly swell when exposed to a fluid (i.e., a pharmaceutically acceptable diluent) and in this swollen form is capable of contributing to a flowable paste that can be applied to a wound site. In some embodiments, the pharmaceutically acceptable diluent is an aqueous solution and may contain a substance selected from the group consisting of NaCl, CaCl₂, sodium acetate, sodium lactate, sodium citrate, sodium caprate and mannitol. For example, a pharmaceutically acceptable diluent comprises water for injection, and—independently of each other—50 to 200 mM NaCl (e.g., 150 mM), 10 to 80 mM CaCl₂ (e.g., 40 mM), 1 to 50 mM sodium acetate (e.g., 20 mM) and up to 10% w/w mannitol (e.g., 2% w/w). In some embodiments, the diluent can also include a buffer or buffer system so as to buffer the pH of the reconstituted dry composition, e.g., at a pH of 3.0 to 10.0, at a pH of 6.4 to 7.5, or at a pH of 6.9 to 7.1.

In some embodiments, each of the first and second hemostatic compositions is liquid absorbing. For example, upon contact with liquids, e.g. aqueous solutions or suspensions (especially a buffer or blood) the hemostatic compositions take up the liquid and will display a degree of swelling, depending on the extent of hydration. Depending on the usage requirements or preferences for the particular hemostatic composition, the hemostatic composition may have a swelling capability from about 38% to about 1600%, from about 300% to about 1600%, from about 400% to about 1300%, from about 500% to about 1100%, or from about 600% to about 900%, by weight. Such equilibrium swell may be controlled, e.g., by varying the degree of cross-linking, which in turn is achieved by varying the cross-linking conditions, such as the type of the crosslinking agent, the duration of exposure of a crosslinking agent, the concentration of the crosslinking agent, the crosslinking temperature, and the like.

Materials having differing equilibrium swell values perform differently in different applications. In some embodiments, the hemostatic composition may be selected according to a desired swelling capability. In some embodiments, the hemostatic composition may be selected according to a reaction parameter selected from the group consisting of a reaction time, a reaction temperature, and a combination thereof. The ability to control crosslinking and equilibrium swell allows the compositions of the present disclosure to be optimized for a variety of uses: while fast swelling may not be desirable in some applications (e.g., neuro-surgery applications), it might be desirable in a trauma/military-type wound.

The present disclosure further provides a method of treating a wound. The method includes selecting a hemostatic composition from a set of hemostatic compositions comprising at least (1) a first hemostatic composition including a first crosslinked polysaccharide selected from the group consisting of cyclodextrin and dextran, wherein the first hemostatic composition has a first degree of crosslinking, and (2) a second hemostatic composition including a second crosslinked polysaccharide selected from the group consisting of cyclodextrin and dextran, wherein the second hemostatic composition has a second degree of crosslinking higher than the first degree of crosslinking. The selected hemostatic composition is administered to a site of the wound.

Although in certain embodiments a dry composition can be directly applied to the target site (and, optionally, be contacted with the pharmaceutically acceptable diluent at the target site, if necessary), it is contemplated to contact the dry hemostatic composition with a pharmaceutically acceptable diluent before administration to the target site, so as to obtain a flowable hemostatic composition in a wetted form, e.g., a hydrogel form. In some embodiments, any of the first and second hemostatic compositions may be mixed with at least one agent selected from the group consisting of a blood clotting factor, fibrin, an antiseptic agent, an anti-microbial agent, a vitamin, a micronutrient, an antibiotic agent, an antifungal agent, prior to being administered to the site of the wound. In some embodiments, at least one additive selected from the group consisting of water-soluble antimicrobial medicines, enzymes, and growth factor agents may be administered with the selected hemostatic composition. The hemostatic crosslinked polysaccharide polymer according to the present disclosure, once applied to a wound, forms an efficient matrix which can form a barrier for blood flow. Specifically, the swelling properties of the hemostatic polymer can make it an effective mechanical barrier against bleeding and re-bleeding processes.

Following are non-limiting examples of hemostatic compositions according to the present disclosure. Persons having ordinary skill in the art will appreciate that variations of the following examples are possible within the scope of the invention, which is defined solely by the claims.

### EXAMPLE 1

β-cyclodextrin was incorporated into crosslinked polymer networks of different crosslinked densities. To obtain a high degree of crosslinking, about 10 g of β-cyclodextrin was mixed with about 10 ml of epichlorohydrin and heated to about 90° C. while stirring in a three-neck 200 ml flask equipped with an about 20 cm-long reverse condenser. A 50% sodium hydroxide solution was added slowly dropwise to produce a whitish precipitate. The heating under intense stirring continued for about 2 hours. After cooling to room temperature, the gelled polymer was spooned out of the flask, repeatedly washed on a Buchner funnel first with distilled water and later with acetone, and dried overnight in a vacuum oven at −30 torr and 50° C. The yield was 80% by weight.

A low degree of crosslinking was obtained using β-cyclodextrin with a crosslinker of poly(propylene glycol) diglycidyl ether, PEG-DGE, (molecular weight=380 a.u.). About 8 g of β-cyclodextrin was mixed with 20 ml of 50% sodium hydroxide and heated to about 130° C. while stirring in a three-neck 200 ml flask equipped with an about 20 cm-long reverse condenser. After 130° C. was reached, 20 ml of PEG-DGE was added dropwise with intense stirring. The precipitate was stirred at 130° C. for about 2 more hours, and about 3 ml of triethylamine was added. The mixture was left stirring overnight. A rubbery gel was obtained after cooling to room temperature. A light brown fraction was removed by repeated wash on the Buchner funnel. The yield was 62% by weight.

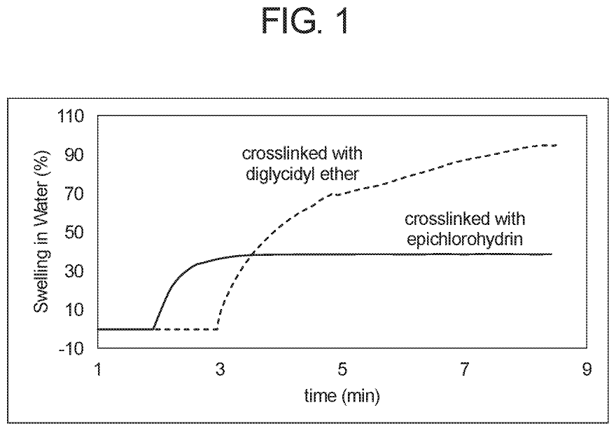

The kinetics of fluid absorption was monitored using thermal mechanical analysis measurement of swelling caused by fluid absorption (TMA). The swelling kinetics was obtained through TMA measurement of the expansion of the resin in the restricted cylindrical volume as a function of time after addition of water to the resin. The experimental results shown in FIG. 1 set forth the following relationship between the level of swelling and the degree of crosslinking in β-cyclodextrin: a high degree of crosslinking with epichlorohydrin causes less swelling, while a low degree of crosslinking with diglycidyl ether causes more swelling. Such a variety of expansion properties can be desirable for allowing clinical personnel to select the absorbent/sealant specific for the wound depth and type.

### EXAMPLE 2

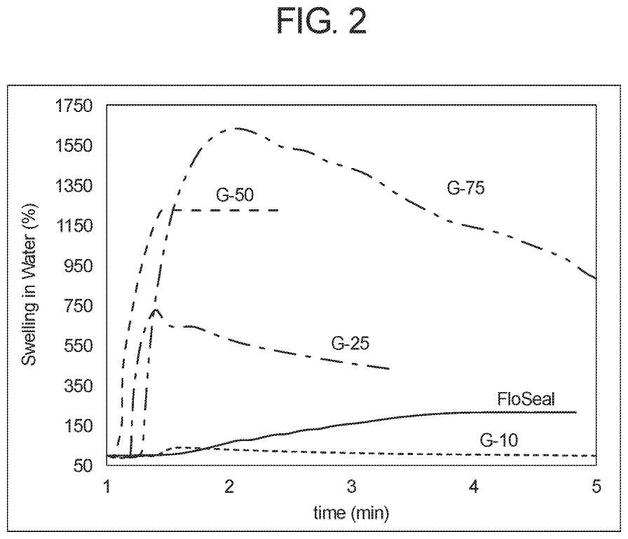

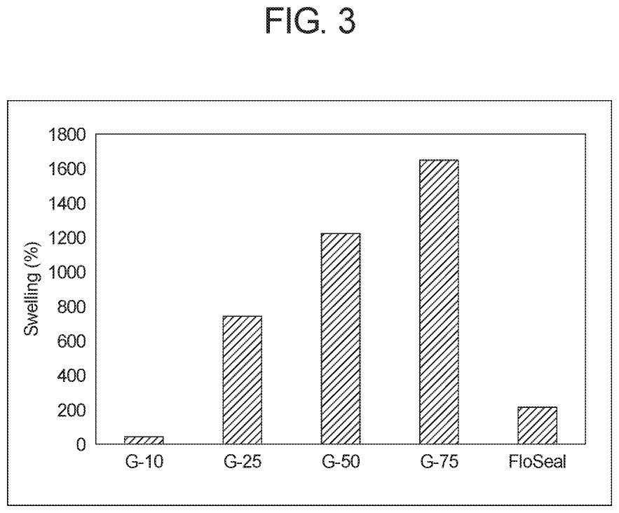

The kinetics of water absorption and the extent of swelling were examined in a series of hydrophilic polymers with a range of crosslinking densities. Sephadex® G-10, G-25, G-50, G-75 crosslinked dextran based polymers from GE Healthcare, were tested. The results shown in FIG. 2 confirmed the control of the water absorption rate and polymer expansion by the extent of crosslinking. Notably, the rate of water absorption by crosslinked dextran polymers Sephadex® (corresponding to the slope of the ascending portion of the kinetic curves of water sorption) appeared to be greater for all tested materials than for the FloSeal gelatin matrix. Table 1 below and FIG. 3 provide the extent of swelling of the Sephadex® polymers relative to that of FloSeal.

TABLE 1
Expansion of materials upon water absorption

| Material | Swelling (%) |
|---|---|
| G-10 | 40.94 |
| G-25 | 744.9 |
| G-50 | 1227 |
| G-75 | 1650 |
| FloSeal | 216 |

The increase in the rate of water absorption by crosslinked dextran polymers relative to FloSeal was unexpected and surprising. In particular, in Sephadex® G-25, G-50, G-75 crosslinked dextran based polymers, a swelling of 744.9%, 1227%, and 1650% was obtained, respectively. In contrast, when FloSeal was used, the swelling was significantly less-216%. The increased rate of water absorption by certain crosslinked dextran polymers can be desirable for rapidly treating bleeding wounds.

Specific embodiments disclosed herein can be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

The invention is claimed as follows:

1. A wound-treating absorbent kit comprising:
a set of hemostatic compositions including at least (1) a first hemostatic composition including a first crosslinked polysaccharide selected from the group consisting of cyclodextrin and dextran, wherein the first hemostatic composition has a first degree of crosslinking, and (2) a second hemostatic composition including a second crosslinked polysaccharide selected from the group consisting of cyclodextrin and dextran, wherein the second hemostatic composition has a second degree of crosslinking higher than the first degree of crosslinking.

2. The wound-treating absorbent kit of claim 1, wherein each of the first and second hemostatic compositions includes crosslinked β-cyclodextrin.

3. The wound-treating absorbent kit of claim 1, wherein each of the first and second hemostatic compositions is in powdered form.

4. The wound-treating absorbent kit of claim 3 comprising a pharmaceutically acceptable diluent for reconstitution of any of the first and second hemostatic compositions.

5. The wound-treating absorbent kit of claim 1, wherein each of the first and second hemostatic compositions comprises a respective crosslinking agent selected from the group consisting of diglycidyl ether, epichlorohydrin, diisocyanate, dicarboxylic acid chlorides, dicarboxylic acid, acid anhydrides, poly(d,l-lactic acid), citric acid, glycerol, dialdehydes, diacyl chlorides, and epoxides.

6. The wound-treating absorbent kit of claim 5, wherein the first and second hemostatic compositions differ from each other in at least one of an amount and a type of the respective crosslinking agents.

7. The wound-treating absorbent kit of claim 1, wherein each of the first and second hemostatic compositions has a swelling capability from about 38% to about 1600%.

8. The wound-treating absorbent kit of claim 1, wherein the second hemostatic composition is configured to absorb less fluid than the first hemostatic composition.

9. The wound-treating absorbent kit of claim 1 comprising at least one additive selected from the group consisting of water-soluble antimicrobial medicines, enzymes, and growth factor agents.

10. The wound-treating absorbent kit of claim 1, wherein any of the first and second hemostatic compositions is mixed with at least one agent selected from the group consisting of a blood clotting factor, fibrin, an antiseptic agent, an anti-microbial agent, a vitamin, a micronutrient, an antibiotic agent, and an antifungal agent.

11. A method of treating a wound, the method comprising:
selecting a hemostatic composition from a set of hemostatic compositions comprising at least (1) a first hemostatic composition including a first crosslinked polysaccharide selected from the group consisting of cyclodextrin and dextran, wherein the first hemostatic composition has a first degree of crosslinking, and (2) a second hemostatic composition including a second crosslinked polysaccharide selected from the group consisting of cyclodextrin and dextran, wherein the second hemostatic composition has a second degree of crosslinking higher than the first degree of crosslinking; and
administering the selected hemostatic composition to a site of the wound.

12. The method of claim 11, wherein the hemostatic composition is selected according to a desired swelling capability.

13. The method of claim 11, wherein each of the first and second hemostatic compositions comprises a respective crosslinking agent selected from the group consisting of diglycidyl ether, epichlorohydrin, diisocyanate, dicarboxylic acid chlorides, dicarboxylic acid, acid anhydrides, poly(d,l-lactic acid), citric acid, glycerol, dialdehydes, diacyl chlorides, and epoxides.

14. The method of claim 13, wherein the first and second hemostatic compositions differ from each other in at least one of an amount and a type of the respective crosslinking agents.

15. The method of claim 11, wherein the hemostatic composition is selected according to a reaction parameter selected from the group consisting of a reaction time, a reaction temperature, and a combination thereof

16. The method of claim 11, wherein each of the first and second hemostatic compositions has a swelling capability from about 38% to about 1600%.

17. The method of claim 11, wherein the second hemostatic composition is configured to absorb less fluid than the first hemostatic composition.

18. The method of claim 11, wherein the selected hemostatic composition is applied in powder form.

19. The method of claim 11, wherein any of the first and second hemostatic compositions is mixed with at least one agent selected from the group consisting of a blood clotting factor, fibrin, an antiseptic agent, an anti-microbial agent, a vitamin, a micronutrient, an antibiotic agent, an antifungal agent, prior to being administered to the site of the wound.

20. The method of claim 11, wherein at least one additive selected from the group consisting of water-soluble antimicrobial medicines, enzymes, and growth factor agents is administered with the selected hemostatic composition.
